(19) United States
(12) Patent Application Publication
(10) Pub. No.: US 2023/0404742 A1
(43) Pub. Date: Dec. 21, 2023

(54) NOVEL REFRACTIVE EDOF INTRAOCULAR LENS FOR CONTINUOUS VISION

(71) Applicant: Biotech Vision Care Pvt. Ltd., Gujarat (IN)

(72) Inventors: Raju Vvnsp PEDAMALLU, Gujarat (IN); Hitendra PATEL, Gujarat (IN); Vinod RATHORE, Gujarat (IN); Vikram PRAJAPATI, Gujarat (IN)

(73) Assignee: Biotech Vision Care Pvt. Ltd., Gujarat (IN)

(21) Appl. No.: 17/994,499

(22) Filed: Nov. 28, 2022

(30) Foreign Application Priority Data
May 25, 2022 (IN) 202221029935

(51) Int. Cl. A61F 2/16

(52) U.S. Cl. CPC A61F 2/1627; A61F 2002/1696

(57) ABSTRACT

The lens of invention has been proven most suitable for the cataract patients. The invention provides extended depth of focus to get continuous vision from far to near distance up to closer distances. The third zone 60 degree segment design of invention is to provide balanced energy distribution at larger pupil diameter and the controlled spherical aberration at each zone provides minimalistic glares and halos. Further the EDOF IOL balances the nominal power and provide pupil independence further optimizing energy distribution in each annular zone to maintain the good contrast irrespective of the pupil size and light condition. Further, zonal modification in the IOL can be used as a controllable switch to change the depth of focus according to the requirement, viz. from distant to near vision or distant to intermediate vision.

### CROSS-REFERENCE TO RELATED APPLICATIONS

This application claims priority to IN Patent Application No. 202221029935 filed on May 22, 2022, the entire contents of which is hereby incorporated by reference.

### FIELD OF INVENTION

The present invention relates to intraocular lens. More particularly the present invention is related to the intraocular lens (IOL) with continuous vision, extended depth of focus (EDOF), and more importantly provides near to distant vision, helps the cataract patients for achieving adequate continuous vision from far distance to near distance objects with minimized halos and glare. Further, zonal modification in the IOL can be used as a controllable switch to change the depth of focus according to the requirement, viz. from distant to near vision or distant to intermediate vision.

### BACKGROUND OF INVENTION

Natural crystalline lens has the ability to adjust its curvature, resulting adjustable focal length according to the distance of the object to be imaged at the retina. However, after the cataract surgery, implanted IOLs perform the same job as the natural crystalline lens but with limited object distances. This is because of its fixed focal length and aperture, which cannot be adjusted according to the object distances as the natural crystalline lens does. Therefore, the person with implanted IOL, cannot see the objects which is beyond the limit of vision distance of the IOL. Thus the person using this type of IOL, has to wear prescription glasses for viewing other distance objects. For example, implanted mono-focal IOL, generally fit for viewing distant objects, hence for intermediate or near objects (for reading purposes) person has to wear prescription glasses. This is because implanted IOL has its fixed focal length and aperture as asserted above.

The present novel invention is an IOL which has an extended depth of focus (EDOF), and can be used to see the near to distant objects and allow the continuous vison without wearing prescription glasses.

The prior art document US20030199976A1 relates to a narrow profile, glare reducing, posterior chamber intraocular lens comprises an optic having an anterior surface and a posterior surface and an optical axis. The posterior surface is formed having two adjacent peri-axial, stepped imaging zones, the two imaging zones having the substantially same optical power. A transition zone between the two imaging zones preferably has a surface of continuous curvature, and shaped to reduce direct glare from light incident on the transition zone of the IOL. In variations, the transition zones are formed at the optic edge to minimize direct and indirect glare in the eye of an individual wearing the intraocular lens.

The prior art document describes the bifocal refractive lens, with an optical power difference between far distance and near distance vision 2.5 D, whereas other prior documents describe the refractive sectorial bifocal lens. These lenses do not have any revolutionary profile.

One of the prior documents describes the bifocal lens with aberration correction, which has been done through counterbalancing the aberration imposed by cornea. Whereas, few other prior documents claim the zonal refractive profile introduced in the IOL, enhances the depth of field up to 1.1 D. The another prior patent describes the use of opaque mask in IOL, having an objective to induce stenopeic effect and hence enhanced depth of field. Various techniques like bulls eye refractive principle of higher power central zones has been used earlier to extend the depth of focus. Further the patent U.S. Pat. No 5,864,380A, document, describe the design of IOL which useful for specifically close range work, while facilitating up to distinct vision up to a specific distance. Another patent document U.S. Pat. No. 9,201,250B2, describes the design which is used in mitigation or treating the refractive error at distances encompassing far to near without significant ghosting.

Few other patent documents describe the opaque mask to induce stenopeic effect, but no inclusion of multifocal surfaces, the variable transmittance with maximum at central region and minimum at periphery of the IOL. It has been claimed in this patent that change in the transmittance allow the lens to increase the depth of field. Yet another prior art also describes the use of phase mask enhances the depth of focus up to 3.0 D. The use of opaque mask in the IOLs compromise the contrast of the image at different light conditions. All these IOLs provide limited solution as described above.

Hence all the above-mentioned prior arts fail to provide a continuous vision IOL with extended depth of focus which is essential in order to view the objects from distant to near with minimal glares and halos.

### SUMMARY OF INVENTION

The present invention relates to intraocular lens. More specifically this invention is related to the intraocular lens (IOL) with continuous vision, along with extended depth of focus (EDOF) which provides continuous vision to the cataract patients from far distance to near distance objects with minimized halos and glare.

One main advantage of the refractive EDOF intraocular lens for continuous vision to provide extended depth of focus to get continuous vision from far distance to near distance around 40-35 cm closer distance. The haloes and glares are minimized by optimizing the spherical aberration at each zone of the anterior surface of the IOL. The lens power and its extended depth of focus are independent from the pupil size or light conditions. Further, zonal modification in the IOL can be used as a controllable switch to change the depth of focus according to the requirement, viz. from distant to near vision or distant to intermediate vision.

The lens of invention has been proven most suitable for the cataract patients. Cataract is an eye disease in which patient vision becomes cloudy because of the natural crystalline lens losses its transparency due to aging effect. During the cataract surgery doctor remove the inner part of the crystalline lens which is responsible for the cloudy vision, and implant the IOL. The implanted IOL provides the similar vision as the natural crystalline lens of the human eye. The present invention is extended depth of focus which provides continuous vision from far distance to near distance along with intermediate distance objects with minimized glare and halos. Further, zonal modification in the IOL can be used as a controllable switch to change the depth of focus according to the requirement, viz. from distant to near vision or distant to intermediate vision.
1. The main objective of the present invention of refractive EDOF IOL is to provide extended depth of focus to get continuous vision from far to near distance up to closer distances.
2. Other main objective of the present invention of refractive EDOF-IOL is the fourth zone 60-degree segment design to provide the balanced energy distribution without compromising the depth of focus at larger pupil diameter like scotopic condition.
3. Another objective of the present invention is that, zonal modification in the IOL can be used as a controllable switch to change the depth of focus according to the requirement, viz. from distant to near vision or distant to intermediate vision.
4. Another main objective of the present invention of refractive EDOF-IOL is to minimize glares and halos by providing controlled spherical aberration at each zone.
5. Further this EDOF-IOL balances the nominal power or distance vision power and provides pupil independence.
6. Another main objective of the EDOF IOL is the optimized energy distribution in each annular zone to maintain the good contrast irrespective of the pupil size and light conditions.
7. Another main objective of the EDOF IOL is to provide the vision at functional intermediate distance and functional near distance at any pupil size irrespective of the light condition, which are missing the traditional multifocal IOLs and monofocal IOLs
8. This lens provides balanced light distribution and maintains the depth of focus along with the image quality and contrast due to the introduction of concentric 60 degree segmented circular zone design.
9. Further this EDOF-IOL anterior surface peripheral zone has been given negative curvature in order to reduce the central thickness. Reduced central thickness is useful during unfold of the lens after passing through the small incision during implantation.

### BRIEF DESCRIPTION OF DRAWINGS OF INVENTION

FIG. 1 represents the anterior surface of the invention.

FIG. 2 represents the posterior surface of the invention.

FIG. 3 represents the main parts of the intraocular lens

FIG. 4 describes the concentric annular zones in the invention.

FIG. 5 describes about the 60-degree arc segments in the fourth zone represented with the part number 7 in FIG. 4 of the invention.

FIG. 6 describes about the posterior surface square edge of EDOF IOL design.

FIG. 7 & FIG. 8 explains about the power distribution/optical output result (autofocus scan) of the invention with optimized circular zones.

### DETAILED DESCRIPTION OF INVENTION

The following paragraphs describes about the design of EDOF IOL and its outcomes and advantages of the design. The subject matter is explained with the drawings at required places, where in like reference numerals are used.

The present invention of intraocular lens is called extended depth of focus Intraocular lens (EDOF IOL) to provide the continuous vision. This lens offers clear vision from far distance to near distance up to closer distances. This new additional feature is providing a near vision along with intermediate vision. Further, zonal modification in the IOL can be used as a controllable switch to change the depth of focus according to the requirement, viz. from distant to near vision or distant to intermediate vision.

The refractive EDOF property is achieved based on the principle of refraction. Refraction is the bending of light ray when passing through one medium to another medium. The bending of light rays is proportional to the curvature of the medium, in which light rays are entering and exit from the medium. Based on this principle the present invention is developed.

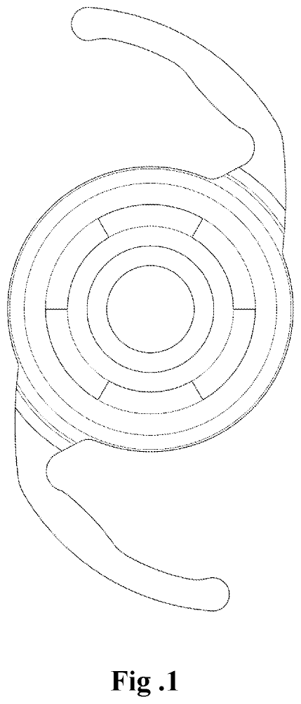

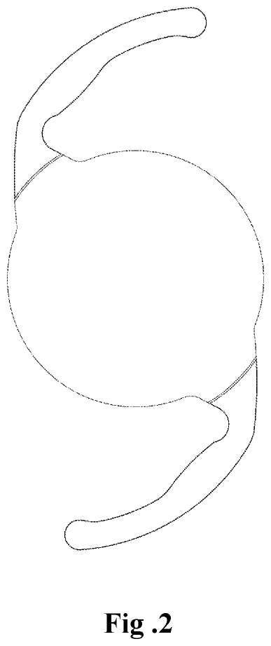

The present invention refractive EDOF IOL is related to intraocular lenses, which are used in the cataract surgery. This refractive EDOF lens is developed with hydrophobic or hydrophilic acrylic material having the refractive index of 1.40-1.6. The EDOF intraocular lens design is having single piece structure as described in the FIG. 1. The front surface of the intraocular lens, called, anterior surface of the invention. This surface is the main objective of this invention, having the controllable refractive circular zones and 60 degree refractive segments in one of the concentric refractive circular zone to provide the continuous vision. The posterior surface of the invention is represented by FIG. 2, have the single radius of curvature for entire posterior surface.

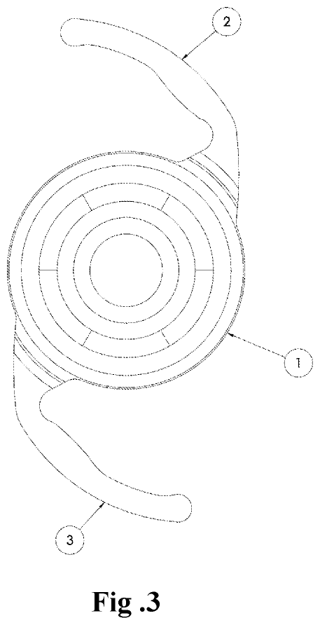

The part number 1 in the FIG. 3 represents the optic portion of the intraocular lens, which is responsible for the light ray refraction and continuous vision inside the eye. The part number 2 & 3 represents the modified C type haptics of the intraocular lens. The modified C type haptics provides maximum contact angle with the capsular bag, which provides the better rotational stability in the eye.

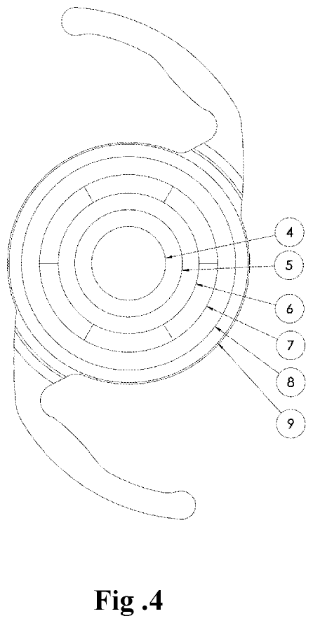

FIG. 4 shows the center optic portion of the intraocular lens having the innovative design to provide extended depth of focus to see the objects from far distance to near distance with continuous vision. The center optic portion having five concentric annular refractive zones represented with the part numbers 4 to 9 as shown in FIG. 4 with in the 4.7 mm clear optic zone on the anterior surface of the optic portion. The peripheral zone as shown in part number 9 has been given negative curvature to the IOL anterior surface to control the thickness of the lens to fold and unfold easily during the implantation of the IOL by using micro incision process.

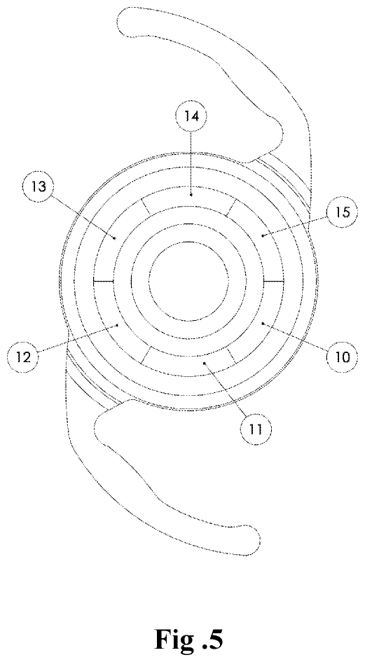

The part numbers 11 to 15 shown in FIG. 5 represents the 60 degree refractive segments in the fourth zone from the center of the invention to balance the energy distribution at larger pupil diameters as shown in the FIG. 4 part number 7. By, zonal modification in the IOL can be used as a controllable switch to change the depth of focus according to the requirement, viz. from distant to near vision or distant to intermediate vision.

Spherical aberration of the lens plays very important role in the optics. When light rays passing through the lens system, paraxial light rays and marginal light rays focus at different focal points on the optic axis, instead of focusing at a single point. In case of negative spherical aberration, marginal light rays refract less and focus far away from the lens surface and in the positive spherical aberration, marginal light rays refract more and focus near by the lens surface. By using this principle, the energy is distributed between the required powers uniformly and provides the extended depth of focus continuously with reduced glare and halos.

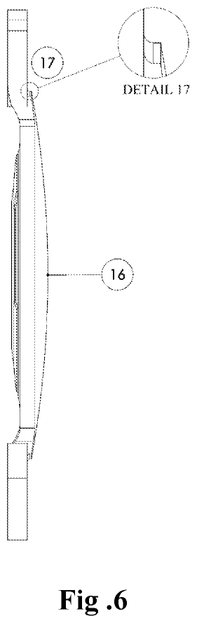

The part number 16 in FIG. 6 represents the posterior surface side view. The part number 17 represents the 360 degree posterior surface square edge of the intraocular lens. The design of the square edge will provide proper shrinkage of the capsular bag with the posterior surface of the intraocular lens and prevent the early stage PCO (Posterior Capsular Opacification).

In the design and development of invention, Hydrophobic or hydrophilic materials are used with variable range of refractive index 1.4-1.6. During the development of the invention, the size of the optic portion has been taken 6 mm with 13.0 mm overall diameter. The optic size and overall diameter of the proposed IOL can be varied as per requirement and it won't affect the optical performance of the depth of focus of the IOL. By using above mentioned methodology, around 3.5 D depth of focus has been achieved by optimizing the zone radius and power distribution on the concentric circular zones. The optimization of the zone diameters and power distribution on the concentric circular zones allow us to achieve desired depth of focus for intermediate distance to near distance visual activities along with far distance.

Optical Material: Hydrophobic or Hydrophilic material

Refractive Index: 1.40-1.6

Optic Surface: Aspheric

Optical Size of the IOL: 6 mm

Number of concentric refractive circular zones in the clear optic area: 5 (in the clear optic zone).

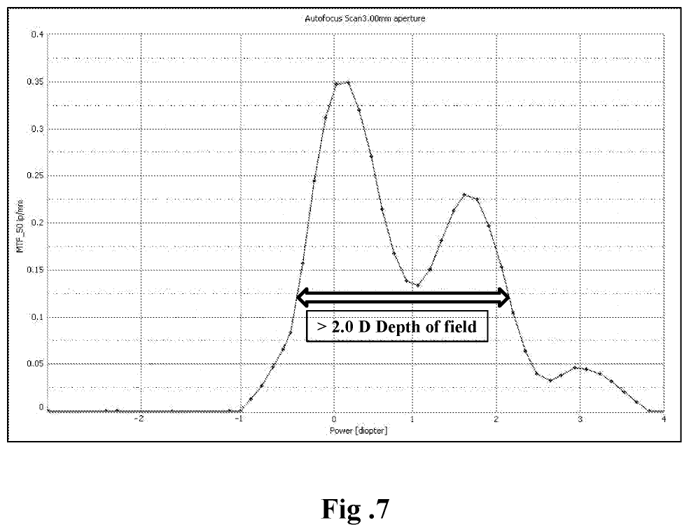

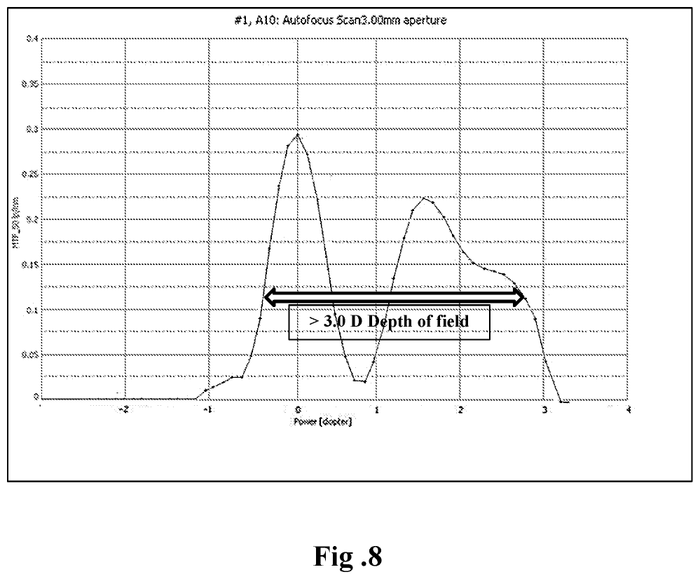

The invention of the Refractive EDOF IOL gives satisfactory results on the optical bench performance. Continuous and extended depth of focus observed in the output result from 1.0 D to 3.5 D. FIG. 7 represents the more than 2.0 D depth of field of the of the intraocular lens by using Modulation Transfer Function (MTF) curve and FIG. 8 represents the more than 3.0 D depth of field of the intraocular lens by using MTF curve. The Modulation Transfer Function (MTF) curve in the FIGS. 7 & 8 shows the energy distribution of the IOL at 3 mm aperture size. The achieved depth of field is useful to see the objects from far distance to intermediate distance as shown in the FIG. 7 and far distance to near distance as shown in the FIG. 8 for daily activities.

The present invention relates to intraocular lens (IOL) with extended depth of focus, which helps in providing adequate vision for far objects to near objects with minimized glares and halos. The main advantage of the present invention refractive EDOF intraocular lens is to provide extended depth of focus to get continuous vision from far distance to near distance. The optimized power distribution on each concentric circular zone and controlled zone width provides the continuous range of vision for daily activities. Controlled spherical aberration and 60 degree refractive segments in the fourth zone makes the nominal power independent from the pupil size (eye pupil) and minimize haloes and glares in the visual acuity of the patient after implanting the lens in the eye.

1. A refractive EDOF IOL comprising:
a tangible circuit configured to and/or capable of providing extended depth of focus to get continuous vision from far to near distance up to closer distances.

2. The refractive EDOF-IOL according to claim 1, wherein said refractive EDOF-IOL or said circuit is a fourth zone 60-degree segment design to be configured to and/or capable of providing a balanced energy distribution without compromising a depth of focus at larger pupil diameter like scotopic condition.

3. The refractive EDOF-IOL according to claim 2, wherein zonal modification in the IOL is used as a controllable switch to change the depth of focus according to a requirement, viz. from distant to near vision or distant to intermediate vision.

4. The refractive EDOF-IOL according to claim 3, wherein said circuit is further configured to and/or capable of minimizing glares and halos by providing controlled spherical aberration at each zone.

5. The refractive EDOF-IOL according to claim 4, wherein said circuit is further configured to and/or capable of balancing nominal power or distance vision power and providing pupil independence.

6. The refractive EDOF IOL according to claim 5, wherein said circuit is further configured to and/or capable of maintaining optimized energy distribution in each annular zone to a predetermined contrast irrespective of pupil size and light conditions.

7. The refractive EDOF IOL according to claim 6, wherein said circuit is further configured to and/or capable of providing the vision at functional intermediate distance and functional near distance at any pupil size irrespective of the light condition, which are missing the traditional multifocal IOLs and monofocal IOLs.

8. The refractive EDOF IOL according to claim 7, further comprising: lens configured to and/or capable of providing balanced light distribution and maintaining the depth of focus along with the image quality and contrast due to the introduction of concentric 60 degree segmented circular zone design.

9. The refractive EDOF IOL according to claim 8, wherein EDOF-IOL anterior surface peripheral zone is given negative curvature by said circuit in order to reduce the central thickness whereby reduced central thickness is useful during unfold of the lens after passing through the small incision during implantation.

10. The refractive EDOF-IOL according to claim 1, wherein zonal modification in the IOL is used as a controllable switch to change the depth of focus according to a requirement, viz. from distant to near vision or distant to intermediate vision.

11. The refractive EDOF-IOL according to claim 1, wherein said circuit is further configured to and/or capable of minimizing glares and halos by providing controlled spherical aberration at each zone.

12. The refractive EDOF-IOL according to claim 1, wherein said circuit is further configured to and/or capable of balancing nominal power or distance vision power and providing pupil independence.

13. The refractive EDOF IOL according to claim 1, wherein said circuit is further configured to and/or capable of maintaining optimized energy distribution in each annular zone to a predetermined contrast irrespective of pupil size and light conditions.

14. The refractive EDOF IOL according to claim 1, wherein said circuit is further configured to and/or capable of providing the vision at functional intermediate distance and functional near distance at any pupil size irrespective of the light condition, which are missing the traditional multifocal IOLs and monofocal IOLs.

15. The refractive EDOF IOL according to claim 1, further comprising: lens configured to and/or capable of providing balanced light distribution and maintaining the depth of focus along with the image quality and contrast due to the introduction of concentric 60 degree segmented circular zone design.

16. The refractive EDOF IOL according to claim 1, wherein EDOF-IOL anterior surface peripheral zone is given negative curvature by said circuit in order to reduce the central thickness whereby reduced central thickness is useful during unfold of the lens after passing through the small incision during implantation.
